(19) United States
(12) Patent Application Publication
(10) Pub. No.: US 2018/0064327 A1
(43) Pub. Date: Mar. 8, 2018

(54) METHOD AND COMPUTING UNIT FOR IMAGING OF WIDER ANGLE FUNDUS OF AN EYE OF A SUBJECT

(71) Applicant: Forus Health Private Limited, Bangalore (IN)

(72) Inventors: Mahabaleswara Ram BHATT, Bangalore (IN); Shyam Vasudeva RAO, Bangalore (IN); Poston TIMOTHY, Bangalore (IN); Anand S. VINEKAR, Bangalore (IN)

(21) Appl. No.: 15/557,471

(22) PCT Filed: May 19, 2015
(86) PCT No.: PCT/IB2015/053668

§ 371 Date: Sep. 11, 2017

(30) Foreign Application Priority Data
Mar. 12, 2015 (IN) 1233/CHE/2015

(51) Int. Cl. A61B 3/00; A61B 3/12; A61B 3/15

(52) U.S. Cl. CPC A61B 3/0025; A61B 3/0041; A61B 3/152; A61B 3/12

(57) ABSTRACT

Embodiments of the present disclosure disclose a method and a computing unit for imaging of wider angle fundus of an eye of a subject. The method discloses guiding placement of an imaging device at set of predetermined locations on the eye. The method further comprises receiving set of images of the fundus of the eye upon determining the imaging device to be at least one of the set of predetermined locations, wherein the set of images are captured by the imaging device upon receiving instructions from the computing unit. The method further comprises determining quality of the set of images and concatenating the one or more images to provide a wider angle target image of the fundus of the eye.

### TECHNICAL FIELD

The present subject matter is related, in general to fundus imaging systems and more particularly, but not exclusively to a method and an apparatus for imaging of wider angle fundus of an eye of a subject.

### BACKGROUND

It is often useful, typically for medical reasons, to examine the fundus of the eye of a human or animal. The fundus is the interior surface opposite the lens, and includes retina, optic disc, fovea, etc. Visible abnormalities in the eye may harm the eye, or reveal the state of other organs of the body.

Most often with a patient able to communicate, such examination involves directing the patient to look at something, standardizing the orientation and state of the eye. The gaze of a small child can be briefly captured by showing an interesting object, but this does not allow for a prolonged study. It is hard or impossible to capture the gaze of an animal or a baby, particularly if premature, or the gaze of a human of any age who is unconscious or otherwise unresponsive, due to trauma. This can make the non-contact scheme used in fundus imaging and in prescribing glasses impractical.

Hence, the medical practitioner needs to apply an ophthalmic coupling gel as an optical bridge between the cornea and lens of the imaging device, and hold the imaging device against the eye. This contact increases discomfort for the patient, and hence there is a need for finishing the procedure quickly. Further, the imaging device holds the eye in position, obviating the need for managing gaze.

Another factor in eye examination is the size of the pupil, which varies with the state of the iris. It is easier to see more through a wider window than a narrow one. In a mature human or infants, the iris enlarges in dim light, therefore eye examinations normally avoid bright lighting. But, this reflex is lacking in a premature baby. Suitable eye-drops enlarge the pupil in most patients, though not necessarily in cases of trauma or drug effects. The drops take up to half an hour to act, and hours to dissipate, and they cause discomfort even in adults. There is thus a range of situations where the fundus must be examined through a narrow pupil. This gives a further reason for contact examination, since one can see more through a small window from very close than from a larger distance.

Even with an imaging system in contact with the cornea, the field of view is limited, as illustrated in FIG. 1. A tip 103 of an imaging device 200 is held near the cornea 150, gaining visual access through the pupil 152 and the opening in the iris 151. A ray 101, 102 enters the imaging device after refraction by the cornea 150 and the lens 156. Refraction at the outer corneal surface can be avoided by including ophthalmic coupling gel, but the effective angle of view 105 between the rays 101 and 102 cannot equal the difference 108 between the angles at which they enter the imaging tip 103. The limited size of the pupil 152 and the convergence properties of the lens 156 constrain imaging of the fundus 158 with the device 103 in a single position. This problem increases with a direct ophthalmoscope, where the imaging device is held near the eye, but not touching or coupled with gel, or an indirect ophthalmoscope, where the imaging device is held further from the eye. If the output is simply an optical image visible to the user, that user needs to be an expert ophthalmologist, who can interpret the output on the spot. This type of examination drastically limits how the results can be recorded and shared.

It is usually required to see the view from more than one direction, so the device 100 is rotated, while neither losing contact with the cornea 150 nor applying unsafe pressure, and remaining aligned on the pupil. This is easier to achieve with a direct control than a mechanical mounting. Where the imaging device 200 is an indirect ophthalmoscope, a head-mounted video camera sharing what the physician sees constitutes a Video Indirect Ophthalmoscope (VIO). Alternatively, one may omit the direct optical view, by including an image sensor in a hand-held device 100, and viewing its output on a fixed display.

Most commonly the immediate output is live video, which enables the user to explore fundus regions of interest by turning the imaging device 200. The view may be directed toward the fovea i.e. central to the fundus where vision is most acute, or to the optic disc i.e. to the nose side of the fovea where glaucoma and other pathologies are most visible, or other points of interest. The user may also sweep through different viewpoints systematically, so as to see everything within a certain distance of the fovea. This is valuable in diagnosis and screening, and the digital output can be stored for comparison, sharing, and so on. A video record of such a sweep, however, requires a large amount of storage, and is cumbersome to search and revisit. It is more helpful to select a set of still images from the video record which between them cover a wide region, and digitally stitch them together to create a single view of that region. Unfortunately, many individual VIO frames have poor quality, and one study reports that only 24% of these videos can be utilized for Retinopathy of Prematurity (ROP) evaluation with ROP tool. Few stills are better, with correspondingly better-stitched wide-angle results, but it remains the responsibility of the user to move smoothly enough, through a wide enough range of directions, for the stitching to cover the desired region. Only a user having long familiarity with retinal geography, and practice in moving a device direction of view around it, can produce adequate results. The cost of such imaging devices is quite high. Further, the usage of such imaging devices is limited due to insufficient pools of expertise.

Manual or automated selection of stills from the video record must be done not only on the quality, but also collective overlap and lacunae. The live or recorded video requires continuous illumination of the retina, and it is harmful for eyes at the developmental stage of a premature infant.

### SUMMARY

In an aspect of the present disclosure, a method for imaging of wider angle fundus of an eye of a subject is provided. The method comprises guiding placement of an imaging device at set of predetermined locations on the eye, receiving set of images of the fundus of the eye upon determining the imaging device to be at least one of the set of predetermined locations, wherein the set of images are captured by the imaging device upon receiving instructions from the computing unit, determining quality of the set of images and concatenating the one or more images to provide a wider angle target image of the fundus of the eye.

In an embodiment of the present disclosure, a computing unit for imaging of wider angle fundus of an eye of a subject is provided. The computing unit comprises a processor and a memory communicatively coupled to the processor. The memory stores processor-executable instructions, which, on execution, causes the processor to guide placement of an imaging device at set of predetermined locations on the eye, receive set of images of the fundus of the eye upon determining the imaging device to be at the set of predetermined locations, wherein the set of images are captured by the imaging device upon receiving instructions from the computing unit, determine quality of the set of images, and concatenate the one or more images to provide a target image of fundus of the eye.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 illustrates an imaging system in contact with the cornea;

FIG. 2A illustrates an apparatus for imaging of wider angle fundus of an eye of a subject in accordance with some embodiments of the present disclosure;

FIG. 2B illustrates an exemplary block diagram of computing unit in accordance with some embodiments of the present disclosure;

FIG. 3 illustrates an exemplary representation of direction in three dimensions, relative to a standard axis in accordance with some embodiments of the present disclosure;

FIGS. 4, 5 and 6 illustrate an exemplary representation of set of directions with different views;

FIGS. 7 and 8 illustrate an exemplary representation of set of directions organized by the symmetry axis of a regular solid in accordance with some embodiments of the present disclosure;

FIG. 9 illustrates an exemplary representation of zones used for ROP in accordance with some embodiments of the present disclosure;

FIG. 10 illustrates an exemplary representation of three views whose overlap includes optic disc in accordance with some embodiments of the present disclosure;

FIG. 11 illustrates an exemplary representation of six views with no point shared by all, but which overlap without holes in their combined coverage in accordance with some embodiments of the present disclosure;

FIGS. 12 and 13 illustrate an exemplary representation of a view of a digital user interface for management of view orientations in accordance with some embodiments of the present disclosure;

FIG. 14 illustrates an exemplary representation of a path through the set of required directions displayed by the user interface in accordance with some embodiments of the present disclosure;

FIG. 15 illustrates an exemplary representation of a view of a digital user interface for management of view directions in accordance with some embodiments of the present disclosure;

FIG. 16 illustrates a flowchart showing a method for imaging of wider angle fundus of an eye of a subject in accordance with some embodiments of the present disclosure;

FIG. 17 illustrates an exemplary representation of geometrical framework for image stitching in accordance with some embodiments of the present disclosure;

FIG. 18 illustrates a block diagram of an exemplary computer system for implementing embodiments consistent with the present disclosure.

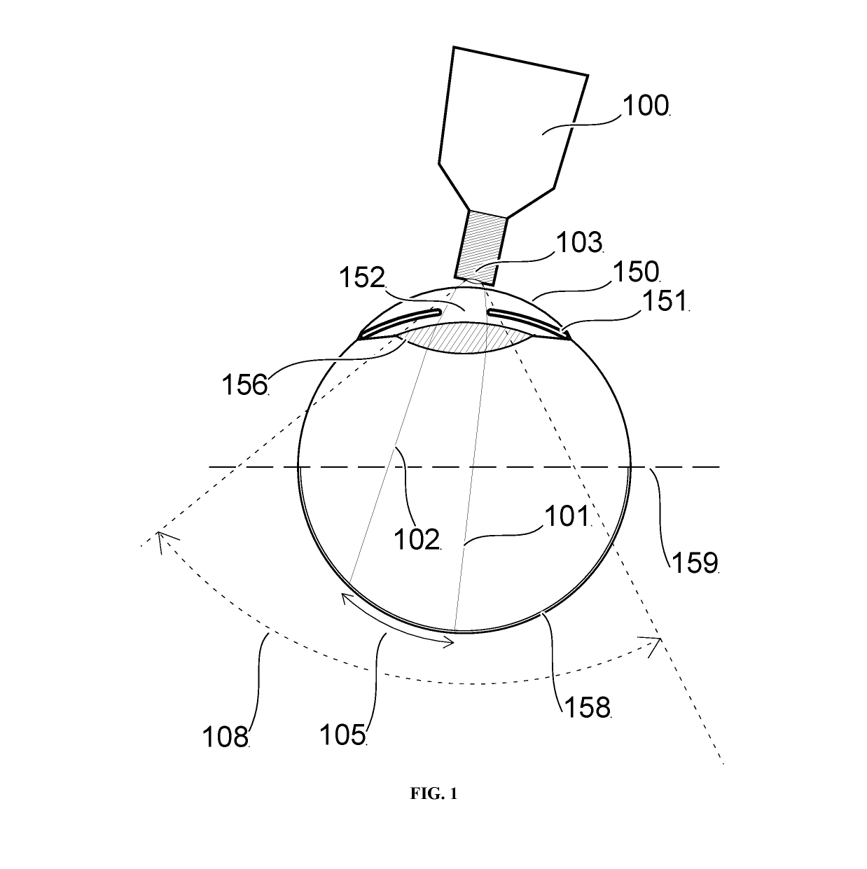

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or apparatus.

Embodiments of the present disclosure are related to a method and an apparatus for imaging of wider angle fundus of an eye of a subject. In particular, the present disclosure relates to a method for capturing images of the retina of an unresponsive subject. The method provides a guidance scheme for using the apparatus. Also, the method provides for constructing there from a wide-angle view of the fundus of the eye.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

The term subject refers to any human being, including an infant, a child, a patient or any other person.

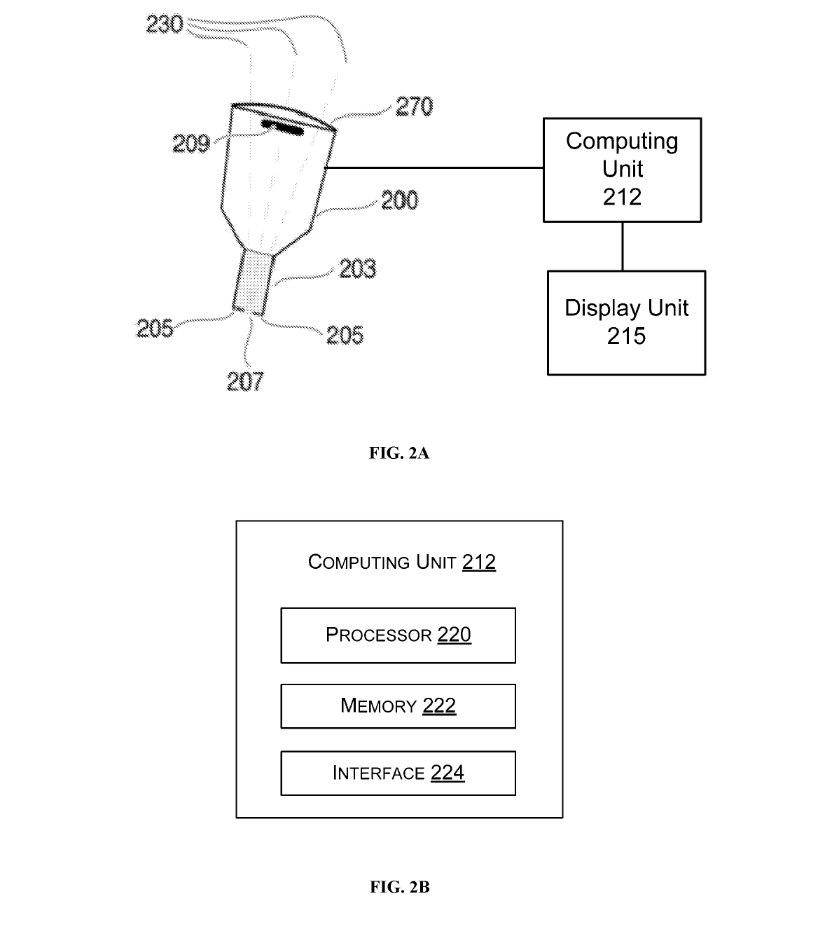

FIG. 2A illustrates an apparatus for imaging of wider angle fundus of an eye of a subject in accordance with some embodiments of the present disclosure. The exemplary environment comprises an imaging device 200 connected to a computing unit 212 and a display unit 215. In an embodiment, the display can be configured within the computing unit 212. In another embodiment, the display unit 215 may be associated to the computing unit 212 externally as illustrated in FIG. 2A.

FIG. 2B illustrates an exemplary block diagram of computing unit in accordance with some embodiments of the present disclosure. The computing unit 212may include at least one central processing unit (“CPU” or “processor”) 220 and a memory 222 storing instructions executable by the at least one processor 220. The processor 220 may comprise at least one data processor for executing program components for executing user- or system-generated requests. A user may include a person, a person using a device such as those included in this disclosure, or such a device itself. The memory 222 is communicatively coupled to the processor 220. In an embodiment, the memory 222 stores information for imaging of wider angle fundus of an eye of a subject. The computing unit 212 further comprises an I/O interface 224. The I/O interface 224 is coupled with the processor 220 through which the input is received.

In an embodiment, the imaging device 200 is a hand-held unit intended to be used near the eye or touching the eye. A tip 203 of the imaging device 200 is shaped to be held against the cornea and coupled to the eye by using ophthalmic gel. The coupling is done for optical advantages and to stabilise the eye. However, a person skilled in the art would understand that uncoupled embodiments are also possible. The hand-held unit 200 includes a light emitting unit 205 of delivering light through the cornea and lens of the eye. The hand-held unit 200 further comprises a light collecting unit 207 for collecting the light reflected from the fundus and directing the collected light to an image sensor 209, which is capable of obtaining still images. The sensor 209 is connected to the computing unit 212 through a communication network (not shown). In an embodiment, the sensor 209 may connected wirelessly or through wired connection to the computing unit 212.The communication network may include, without limitation, a direct interconnection, local area network (LAN), wide area network (WAN), wireless network (e.g., using Wireless Application Protocol), the Internet, etc.

The imaging device 200 receives instructions for capturing images from the computing unit 212.Then, the imaging device 200 transmits the captured images to the computing unit 212 for further processing. In an embodiment, the image sensor 209 captures still images, of high resolution and quality. In an embodiment, the image sensor 209 may also capture a video. As an example, the image sensor 209 may be hyper-spectral sensor.

The images may be acquired with the imaging device 200 which is held still for each imaging, or at moments selected by the computing unit 212. The moments are selected by the computing unit 212 by continuous, un-paused motion of the imaging device 212, when the device is detected to be at an appropriate position.

In an embodiment, the imaging device 212 is moved by a human user. The method of present disclosure provides guidance to user on to go the selected positions as successive static locations, or to move through continuously them. Also, a feedback is provided when a re-take is needed.

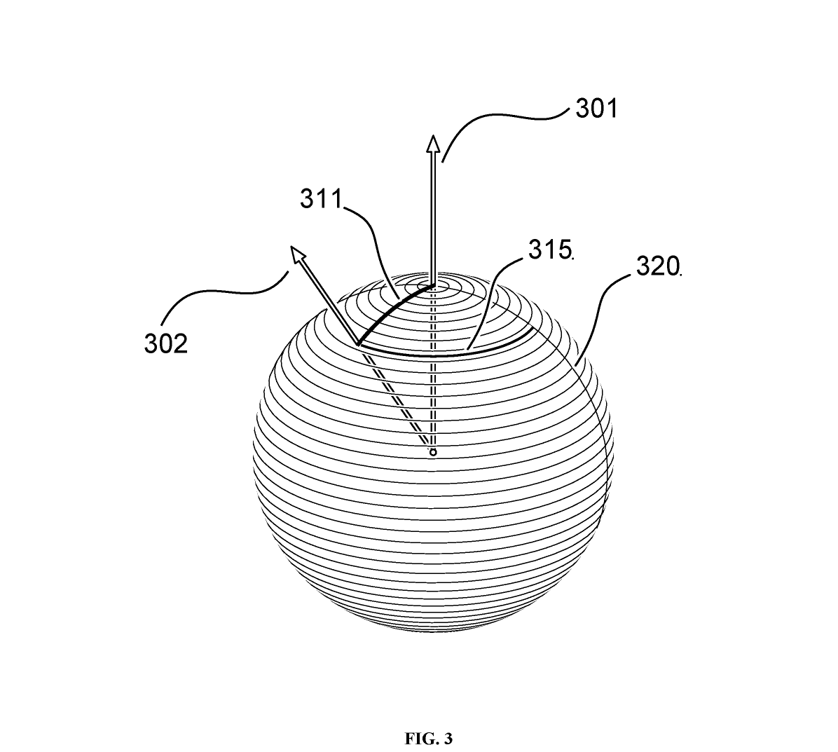

FIG. 3 illustrates an exemplary representation of direction in three dimensions, relative to a standard axis in accordance with some embodiments of the present disclosure. The imaging device 200 includes a set of preferred directions 230, defined relative to the imaging device 200, which are aligned with the eye axis when corresponding still images are to be taken. In an embodiment, the present disclosure includes sensors, markers and other known methods that guide the user in achieving such alignment. These directions do not fully define the orientations required for the imaging device 200, but they constrain it, as follows.

Suppose that the patient's eye is directed vertically upward. The eye axis then corresponds to the vertical arrow 301 in FIG. 3, with the centre of the sphere taken as the point of contact with the cornea. A preferred direction 230 may be made to coincide with this axis, by holding that direction vertical. The arrow 302, pointing back along the axis of the imaging device 200 then makes a particular angle 311 with the eye axis direction 301.This does not fully determine the view obtained by the imaging device 200, which is changed by rotation about either the vertical axis 301, or the unit axis 302. The rotation about the unit axis 302 does not greatly affect the part of the retina seen by the imaging device 200, since it merely spins the view about its centre.

In the image acquired by the imaging device 200 what is seen through the iris is approximately a disk centred on the ‘straight forward’ point of the imaging device 200, so that at all points of interest a rotation in software can compensate for rotation about the unit axis.

In an exemplary embodiment, one direction 302 is along the eye axis, with the others grouped around it. Each axis direction gives a set of through-iris visibility directions, which is approximately a cone centred at the tip 103 of the imaging device 200. The focusing effect of the eye leads to a more complicated dependence of visibility on the orientation of the unit, but for clarity of exposition, a common centre for all views is considered. Further, it is considered that through-iris visibility exists in a right circular cone whose axis is the optical axis of the imaging device 200.

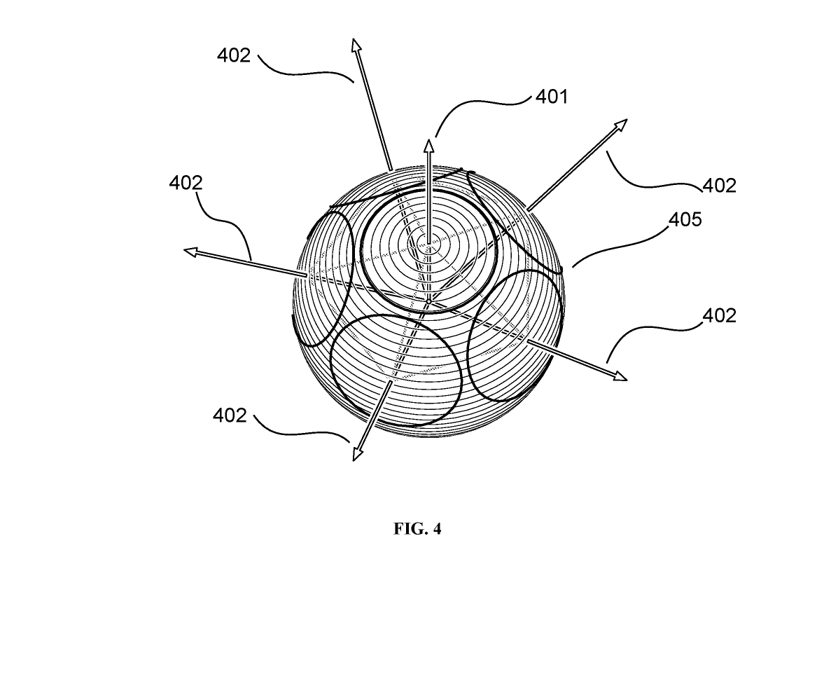

The present disclosure requires images of retinal regions that overlap to create a larger region without gaps, but the approximation allows us to consider cones of visibility from a shared point, and their overlaps. These may be visualised using the boundary circles of their meeting with a unit sphere. FIGS. 4, 5 and 6 illustrate an exemplary representation of set of directions with different views. FIG. 4 shows a central direction 401, surrounded by directions 402, from a common point. It is to be noted that this is not the centre of the eyeball, but reference point in showing directions. Pointing the imaging device 200 in one of these directions enables the imaging of retinal points in directions within a cone angle ‘α’ of that direction, pointing through a corresponding circle 405 around that axis. In this illustration, the angle ‘α’ is less than half the angular separation ‘β’ between the directions 402, so that there is no overlap and the set of visibility directions is disconnected. It follows that the set of visible retina points, at whatever distance each lies along its visibility direction, is also a disconnected set, and cannot provide the combined view needed for medical study.

It is not sufficient, however, to ensure that the angle between viewing directions satisfies the condition below:

β≦2α (1)

FIG. 5 illustrates that this condition ensures the necessary overlaps 555 between pairs of retinal windows, but gaps 566 remain between neighbouring triples. The general condition to avoid gaps between three directions which are pair wise an angle β apart, is that

$$cos β \geq \frac{( 3 ( {( cos α )}^{2} ) - 1 )}{2} , \qquad ( 2 )$$

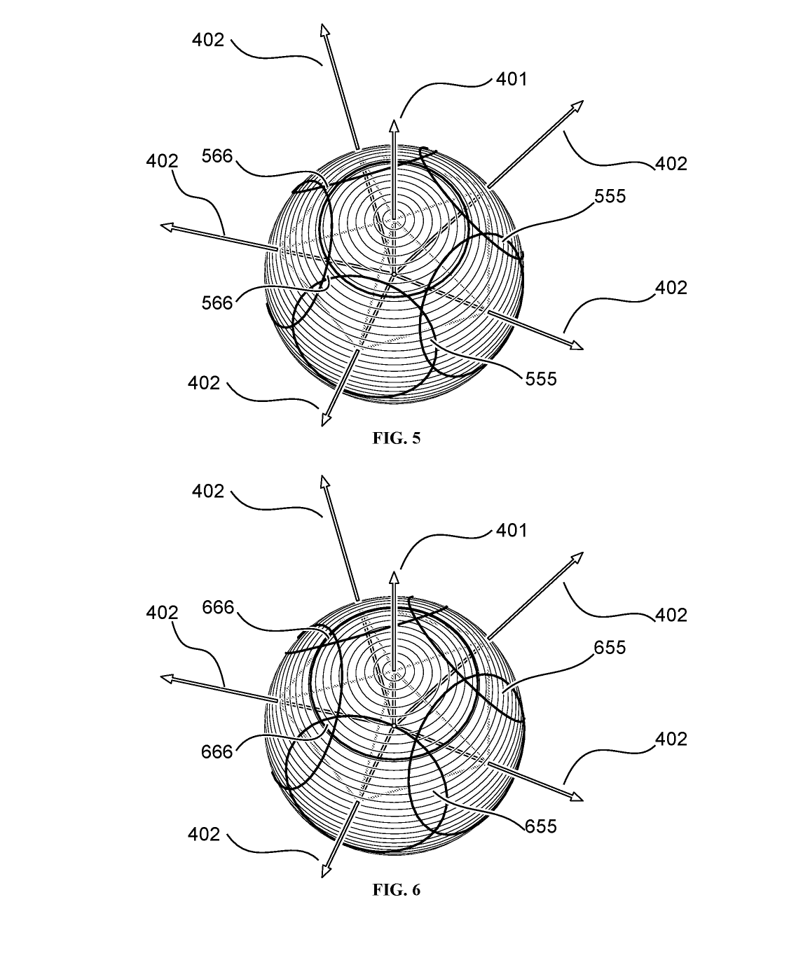

which holds in FIG. 6. With a larger cone angle, the angular radius of the visibility circles, the gaps 566 are replaced by triple overlaps 666. It is not necessary that these overlaps be large, i.e. an overlap 555 between two nearest-neighbour retinal windows should contain enough identifiable points for good matching when the images are stitched, but a triple overlap 666 is required only to avoid omission of retinal points. The stitching is defined by the pair wise overlaps.

For small angles, condition (2) above requires approximately that

β≦α\sqrt{3} (3)

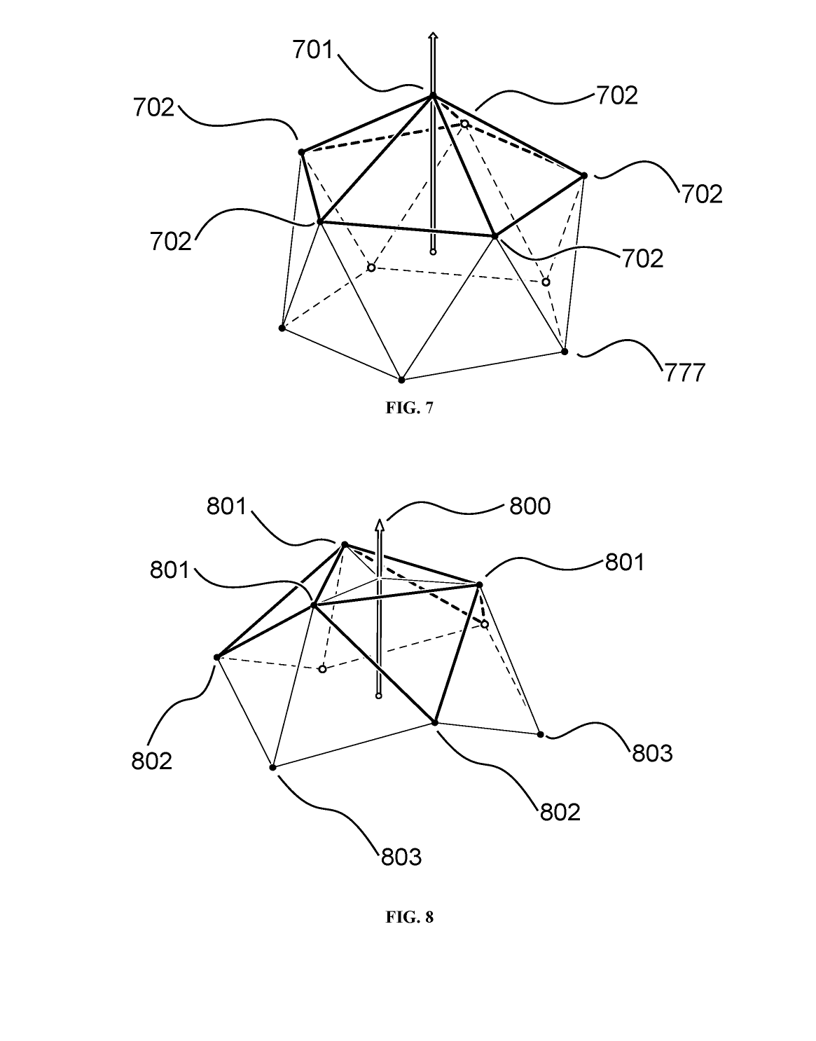

The condition is like the relation between the sides of an equilateral triangle and the distance sufficient to cover the centre from a corner. However, on the sphere of possible directions, there are only a few networks of points with equiangular triangles, and the angles are not small. The points are necessarily vertices of a regular polyhedron, of which only the tetrahedron, octahedron and icosahedron have triangular faces. The smallest angle β between neighbouring vertices, measured from the centre, is approximately 63.9°, and equation (2) requires that the visibility cone angle α should be at least 37.38°. If a particular design for the unit enables such an ‘α’, six of the twelve vertex directions of the icosahedron can be used (the other six being the same ones, backward). FIGS. 7 and 8 illustrate an exemplary representation of set of directions organized by the symmetry axis of a regular solid in accordance with some embodiments of the present disclosure. The obvious way to do this is to make the direction of one vertex 701 central, in FIG. 7, and its five neighbours 702 grouped around it. In the current approximation, the combined coverage is more than the entire rear half of the eye. In practice, the difficulties of an oblique view through the iris and lens would severely limit the outermost view in a direction 702, and reduce its effective angle ‘α’ inward to the axis 701. The criterion (2) is treated as a preliminary guide in design, to be supplemented by detailed optical model of the eye and image acquisition unit.

An alternative use, FIG. 8 of the icosahedral form is to group three neighbouring vertices 801 symmetrically around a central ‘forward’ direction 800. However, the shared neighbours 802 of the vertices 801 point nearly at right angles to the central direction 800, and the unshared neighbours 803 actually point backward, so none of these are likely to be useful in retinal imaging. If only the three vertices 801 are used, a tight cluster with limited total coverage is obtained. In an embodiment, with only a single triangle the existence of a larger pattern of equal angles between directions is not required, as in the icosahedron, and the triangle may be made larger or smaller as long as (equation 2) is satisfied, and a small ‘α’ compels shrinkage. Similarly, the ring of five directions 702 may be shrunk toward the central direction 701, but the resulting spherical triangles are no longer equilateral. For high shrinkage, this would be best approximated with a ring of six directions, like the six neighbours of a point in a hexagonal grid. The equation 2 must be replaced by a less symmetrical criterion, as will be evident to one skilled in the art. A substantially smaller ring could be surrounded by a ring of further directions like 777, without their pointing backward relative to 701, but this only becomes necessary or useful if ‘α’ is small and a wide stitched coverage is needed.

Different selections of direction sets are appropriate in different embodiments of the present disclosure, because different medical goals give rise to different needs both in the size of the retinal region imaged, and in the positioning of it. In some contexts, the most important area of the retina is the fovea, where the sensitivity to detail is most acute and the surrounding macula. The fovea lies opposite the eye's iris and lens, so one may centre an image on it by alignment with the eye axis. As an example, for a composite image, aligning 701, 800 or analogous directions with it. Further, the macula may be successfully imaged by a three-region overlap. For a wider view, a set of five or six directions around a central direction like FIG. 7 gives a more evenly round combined region.

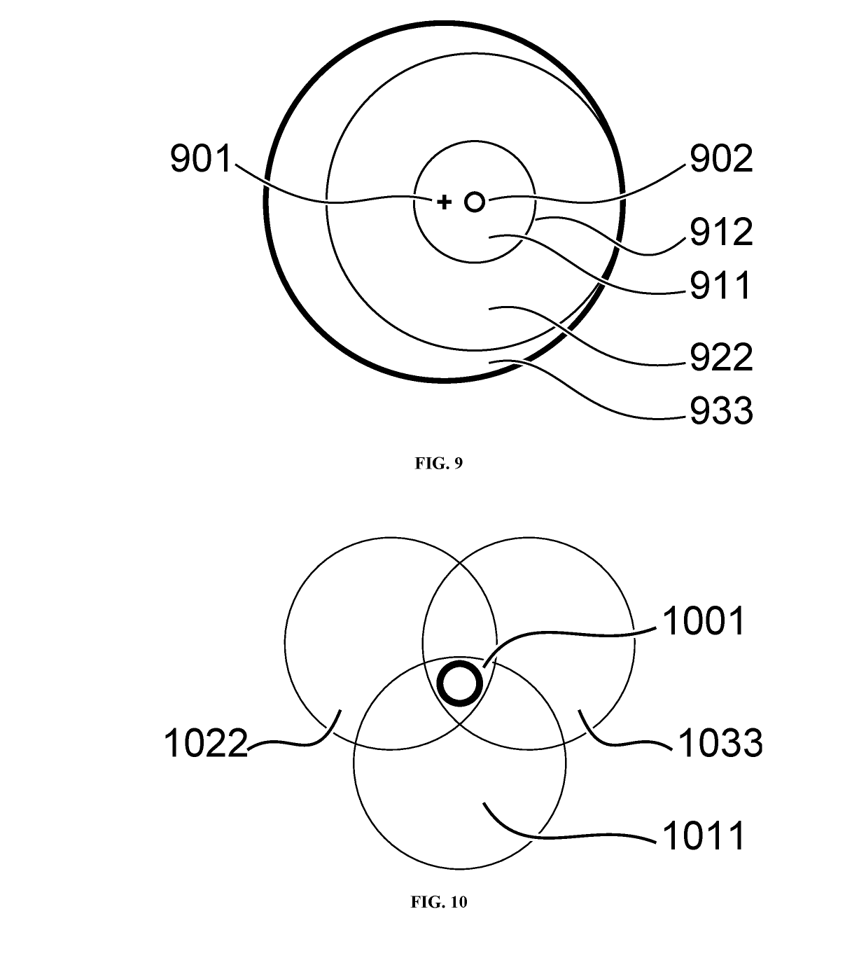

FIG. 9 illustrates an exemplary representation of zones used for ROP in accordance with some embodiments of the present disclosure. For some conditions, however, the fovea is not the appropriate imaging centre. ROP involves growth errors in the blood vessels across the retina from the optic disk, via which the vessels and nerves connect with the outside of the eye, and cause the absence of receptors known as the ‘blind spot’. This lies nearer to the nose. Since growth begins there, the medical terminology for ROP takes it as a centre. As illustrated in FIG. 9, Zone I, i.e. the innermost zone, 911 comprises of a circle 912, the radius of which extends from the center of the optic disc to twice the distance from the center 901 of the optic disc to the center 902 of the macula. The retinal area 922 defined as zone II extends centrifugally from the edge of zone I to the nasal or a serrata (at the 3-o'clock position in the right eye and the 9-o'clock position in the left eye). Zone III is the residual crescent 933 of retina anterior to zone II. Zone 1 consists of the interior 911 of the circle 912, not only the curve 912. The term circle could refer to points equidistant from the disc centre 902, with distance measured in the retinal surface, or measured instead in their optical projection to a flat image. These are not equivalent measures, and indeed unless the optical system is equivalent to a pin-hole camera whose hole is in the spherical surface occupied by the retina, a retinal circle becomes an image ellipse, and vice versa. Even for a pin-hole image, circles concentric on the sphere are eccentric on the flat image, and twice the distance is similarly imprecise.

Even within the context of ROP, then, the medical need may be for a combined image of part of the retina, centred on the optic disc, or for a wide overview that includes Zone III and for efficiency in acquisition should centre on the fovea. Correspondingly, the direction set to be used may be symmetrical about an axis through the optic disk or an axis through the fovea, with or without alignment of one of the set directions with the symmetry axis FIGS. 7 and 8 illustrate these two embodiments. Moreover, since for the two eyes the direction toward the optic disc leans to different sides of the central axis, the two sets of directions centred around it differ as absolute spatial directions. As these examples illustrate, there is a need for a single system that manages multiple direction sets.

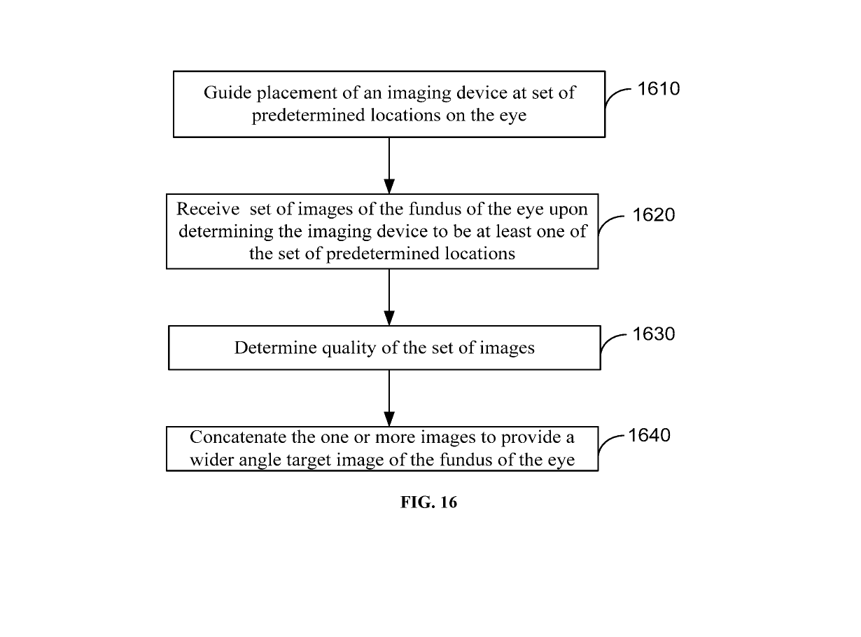

FIG. 16 shows a flowchart illustrating a method for imaging of wider angle fundus of an eye of a subject in accordance with some embodiments of the present disclosure.

As illustrated in FIG. 16, the method comprises one or more blocks for imaging of wider angle fundus of an eye of a subject. The method may be described in the general context of computer executable instructions. Generally, computer executable instructions can include routines, programs, objects, components, data structures, procedures, modules, and functions, which perform particular functions or implement particular abstract data types.

The order in which the method is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

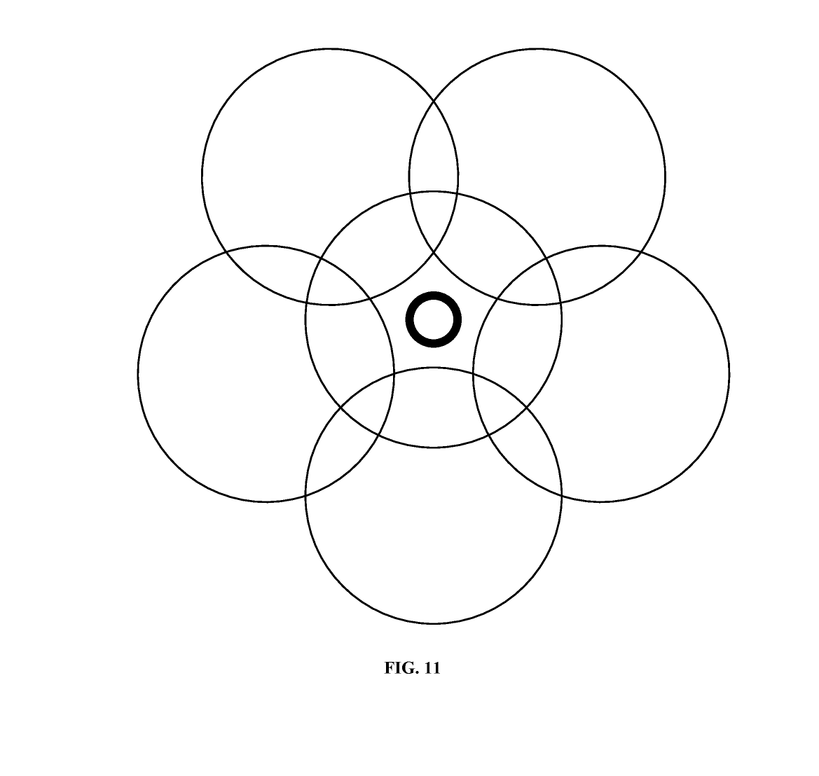

In an embodiment, the subject is placed in a supine position, with gaze upward. A user of a device following current art needs considerable experience with retinal imaging to choose directions by what is seen through the device. FIG. 10 illustrates an exemplary representation of three views whose overlap includes optic disc in accordance with some embodiments of the present disclosure. As illustrated, a set of three around Zone 1, arranged as in FIG. 8, could be achieved by choosing one view 1011 with the optic disc visible 1001 at the top of the displayed image (in clock terms, the 12 o'clock position), and the others 1022 and 1033 with the optic disc at respectively the 4 o'clock and 8 o'clock positions. Even in this case, the user locates the disc first, then swivels the imaging device 200 to the corresponding directions. The overlap on the highly visible feature 1001 ensures adequate data for stitching the three views into one. However, it is harder to achieve the similarly sized views in FIG. 11 (corresponding to viewing axes as in FIG. 7, if 701 points to the optic disc), which combine to a wider image.

Referring back to FIG. 16, at block 1610, guiding, by the computing unit 212, placement of the imaging device 200 at set of predetermined locations on the eye. In an embodiment, the present disclosure guides the user without reference to the current view of the retina. The guidance may be provided by various known methods including, but not limited to reflection, gravitation and digital technique. Further, the placement of the imaging device can also be determined by at least one of position of a bolus of fluid moving within another fluid of different density, position of a reflection on a specular surface, output of at least one accelerometer mounted on the device, computation from electromagnetic interaction, computation from optical measurements, computation from ultrasound measurements etc. A person skilled in the art would understand that any other methods may be used with the method of the present disclosure.

The process of guiding the placement of the imaging device 200 using reflection method is described herein. In an embodiment, a detachable curved reflective surface 270 is added to the imaging device 200, and a target light or other bright source is included at a fixed point in the environment. If the viewpoint of the user of the imaging device 200is fixed at another point in the environment, the point on the surface 270 at which the user sees the reflection of bright source depends on the orientation of the imaging device 200. If the user moves the imaging device 200 (maintaining contact of the tip 203 with the eye) to make this reflection point coincide with a marking on the surface 207, this constrains the possible orientations of the imaging device 200. If further the bright source has a visible direction (for instance, as a short narrow strip of light), and the marking on the surface similarly has a direction, aligning the reflection and its direction with the marking completely determines the orientation of the imaging device 200. A set of such directional markings, each corresponding to an orientation of the imaging device 200 produces a view in the desired set of directions, thus enables the user to place the imaging device 200 in each of such orientation in turn. A change of the surface 270 to one with different markings enables a different set.

Another technique for guiding the placement of the imaging device 200 is using gravitation technique. The curved surface 207 may be transparent and displays a bubble beneath it, in the manner of a spirit level. For each orientation, a particular point on the surface 207 is highest, so that the bubble moves to that point.

Another alternative embodiment for guiding the placement of the imaging device 200 is using digital technique. The position and orientation of the imaging device 200 may be found at successive moments by digital processing of electronically acquired data. For example, one or more digital video cameras may acquire fixed-viewpoint images of an object with landmark features, from which algorithmic recognition enables computation of the location and orientation of the imaging device 200. In another example, sensors within an object may measure its translational and rotation acceleration, enabling numerical integration to deduce its current location and orientation. Any system for achieving this, current or developed in the future, may be used within the embodiments of the present disclosure.

In an embodiment, the computing unit 212 can display the current orientation of the imaging device 200, relative to a target orientation to the user. In such embodiment, the computing unit 212 continually recalculates the location of the tip of the imaging device 200. The tip should be on the cornea throughout the procedure. Any substantial change signals that the imaging device 200 has slipped or the tracking system is wrong, both of which is signaled to the user as a fault.

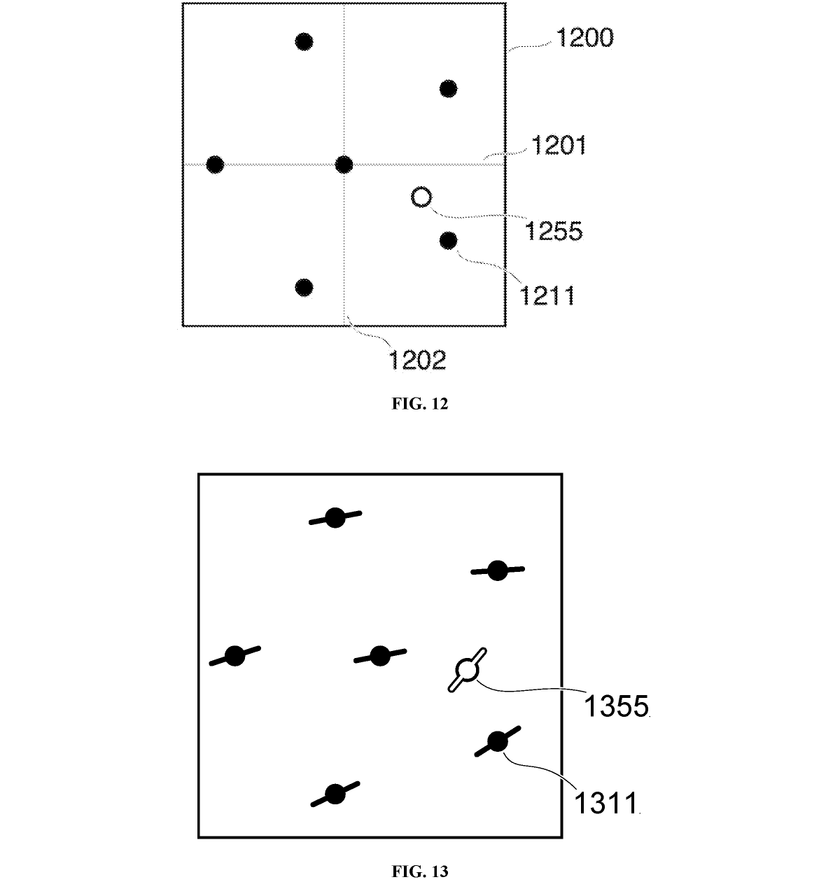

FIG. 12 illustrates an exemplary representation of a view of a digital user interface for management of view orientations in accordance with some embodiments of the present disclosure. In an exemplary user interface, an orientation may be displayed by computing unit 212. The orientation comprises (x,y) point at which the optical axis of the imaging device 200 intersects a horizontal plane Pat a fixed height above the eye. Using an x-axis 1201 and a y axis 1202, which may or may not be shown on the display 1200, a fixed mark 1211 is shown at each point corresponding to an axis in a desired direction Also, a mobile mark 1255 is shown at the point corresponding to the current optical axis of the imaging device 200. When the mark 1255 is centred on a fixed mark 1211, the imaging device 200 can capture the corresponding view. In an embodiment, the optical axis direction of the imaging device 200 needs to be within a specified tolerance of the desired direction. The mark 1255 may be watched while moving the imaging device 200 to align the imaging device 200as required. Optionally, a mark 1211 can be augmented with an indicator 1311, as in FIG. 13, showing the direction in which a particular plane fixed in the imaging device 200 and containing its optical axis, should meet the plane P. The mobile mark 1355 has a corresponding direction indicator, which turns as the imaging device 200 revolves about its optical axis. Aligning the oriented mark 1355 with a mark 1311 brings the imaging device 200 into a completely specified orientation. This may be useful if, for example, the field of view of the imaging device 200 is not effectively circular around its optical axis.

At block 1620, receiving, by the computing unit, set of images of the fundus of the eye upon determining the imaging device to be at least one of the set of predetermined locations. In an embodiment, the set of images are captured by the imaging device 200 upon receiving instructions from the computing unit 212.

In an exemplary embodiment, since the computing unit 212 can assess whether the imaging device 200 is in an appropriate position, it is not necessary for the user to take a separate action to trigger the acquisition of an image. The computing unit 212 can highlight each mark 1211 or 1311 in turn, wait until the imaging device 200 is within tolerance of the required direction, acquire an image, and highlight the next mark. If the image is acquired by a brief flash, motion artefacts may be absent; so that the user needs only to make the marker 1455 follow a curve sufficiently close to a trajectory 1400 that each mark 1411 is visited in turn. This can be done faster than by watching video output of a retinal image, with the need to fixate on landmarks individual to the subject, and it can be trained without the need of an artificial eyeball with an internal pattern.

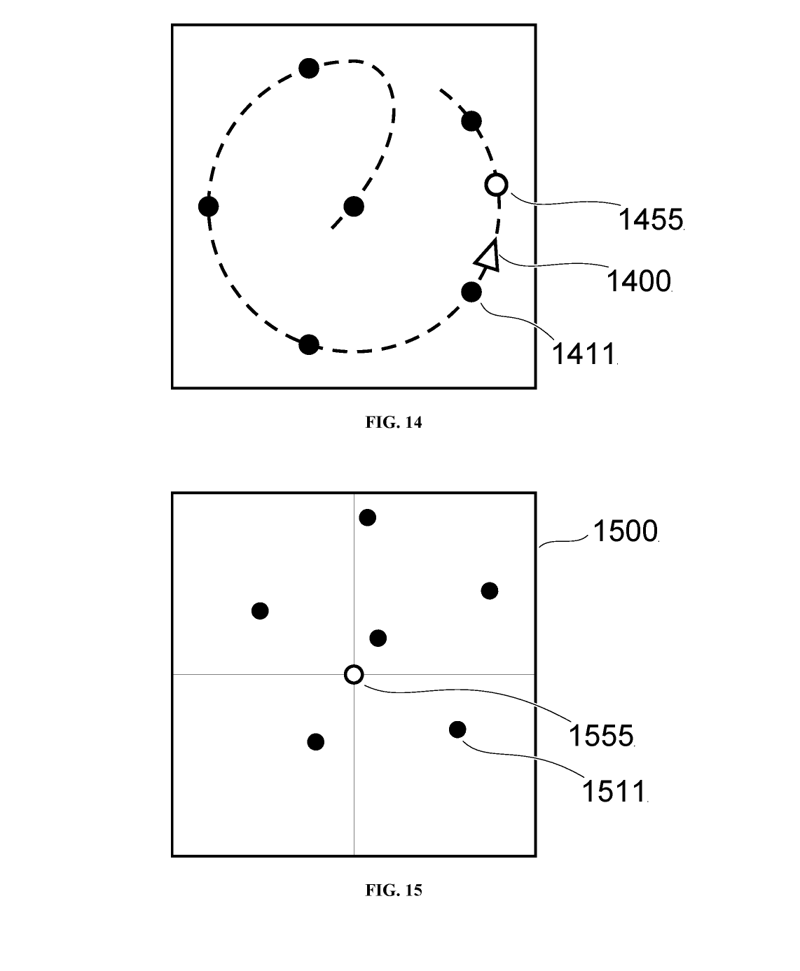

In the exemplary embodiment illustrated in FIGS. 12, 13 and 14, the computing unit 212 provides a display in which the target marks are fixed, and the current position mark is mobile. In an embodiment, a flat display 1500 is attached to the top of the imaging device 200. In the display 1500, a marker 1555 for the axis direction is centrally fixed. The target direction markers 1511 translate and rotate according to the current positions of the desired directions in the reference frame of the imaging device 200. This enables the user to direct hand and eye attention to the location of the imaging device 200 itself reducing cognitive and ergonomic load.

In an embodiment, the user of the imaging device 200 selects a region, such as “Zone 2”, to be imaged, and a choice of left or right eye, which determines the direction set to be used. As described above, the patient is placed in a supine position, with gaze upward. The user places the imaging device 200 against the cornea of the chosen eye, optically coupled to it by gel.

The computing unit 212 in or connected to the imaging device 200 uses a display, on the imaging device 200 or separate, to show markers corresponding to the current orientation and to those in the selected direction set, with the first one highlighted. While the user moves the imaging device 200, maintaining contact with the eye, the computing unit 212 repetitively tests whether the optical axis of the imaging device 200 is within a tolerance ‘ε’ of the next required direction (and, optionally, a required angle around that direction). If it is not, the highlight remains on the current target option while the user continues to move the imaging device 200 in a direction intended to bring its axis closer to the target. If it is within tolerance, the computing unit 212 triggers a flash and acquires the corresponding image.

At block 1630, determining, by the computing unit, quality of the set of images. If quality of the image is not adequate, the highlight remains on the current target option, and the user continues to handle the imaging device 200 with the same target, until the image quality is acceptable. If the image quality is adequate, the computing unit 212 checks 1609 whether the remaining directions are covered. If all the directions are not covered, the computing unit 212 advances to the next target and highlight it. In the alternative, if all the directions are covered, the method proceeds to block 1640.

At block 1640, concatenate, by the apparatus, the one or more images to provide a wider angle target image of the fundus of the eye. The separate images overlap in their coverage of fundus regions. The computing unit 212 stitches the images together into a single wide-angle fundus image.

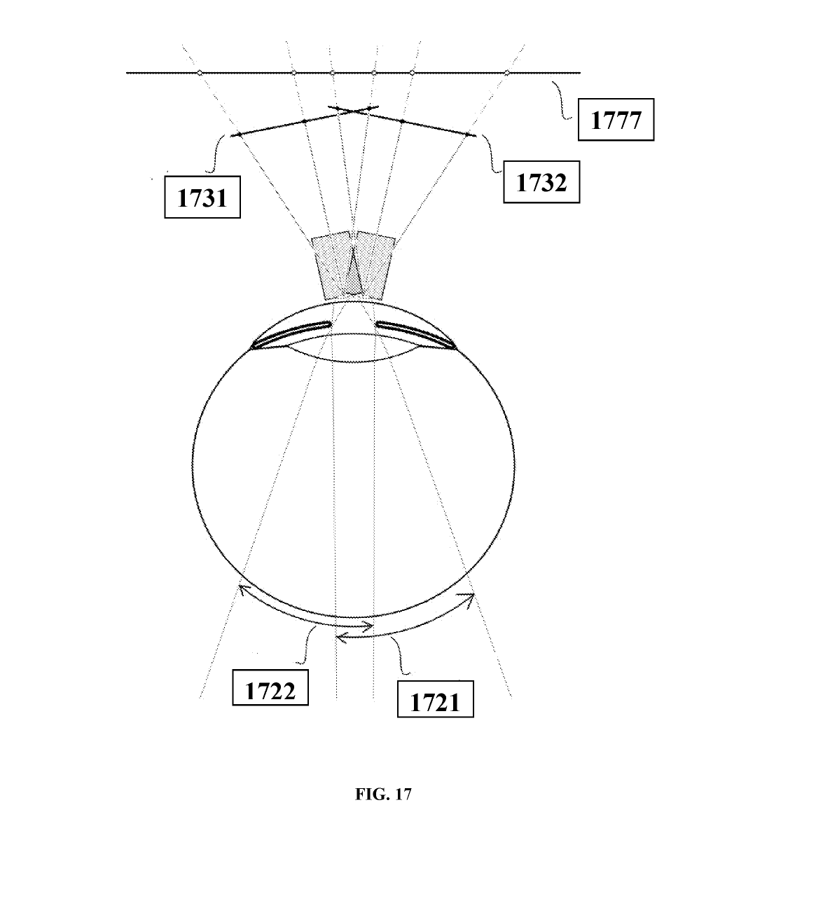

Each image is non-linearly distorted, relative to the spherical shape of the retina. More importantly, it is non-linearly distorted, relative to each other image. FIG. 17 illustrates this with two positions for the imaging device 200, giving two retinal fields of view 1721 and 1722. The images projected respectively onto flat back plates 1731 and 1732, which for clarity the drawing enlarges beyond the physical space of the imaging device 200. There are retinal points which are imaged on both plates, so that a sub-region of 1731 and 1732 correspond by the relation ‘imaging the same point’, but the correspondence is not linear, let alone a rigid, jig-saw-like relation allowing one to be rotated and slid onto the other for a perfect stitching match. The desired output is a flat image on a formally existing single flat plate 1777, which includes the images in 1731 and 1732 and shows as single points the points that both see; but since these images are not linearly related, the image on the formal 1777 plate cannot be linearly related to more than one of them. In a symmetrical approach, it is not linearly related to either (or with three or images, any) of them.

### Computer System

FIG. 18 illustrates a block diagram of an exemplary computer system 1800 for implementing embodiments consistent with the present disclosure. In an embodiment, the computer system 1800 is used to implement the computing unit 212. The computer system 1800 determines breach of security in an organization. The computer system 1800 may comprise a central processing unit (“CPU” or “processor”) 1802. The processor 1802 may comprise at least one data processor for executing program components for executing user- or system-generated business processes. A user may include a person, a person using a device such as such as those included in this disclosure, or such a device itself. The processor 1802 may include specialized processing units such as integrated system (bus) controllers, memory management control units, floating point units, graphics processing units, digital signal processing units, etc.

The processor 1802 may be disposed in communication with one or more input/output (I/O) devices (1811 and 1812) via I/O interface 1801. The I/O interface 1801 may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n /b/g/n/x, Bluetooth, cellular (e.g., code-division multiple access (CDMA), high-speed packet access (HSPA+), global system for mobile communications (GSM), long-term evolution (LTE), WiMax, or the like), etc.

Using the I/O interface 1801, the computer system 1800 may communicate with one or more I/O devices (1811 and 1812). For example, the input device 1811 may be an antenna, keyboard, mouse, joystick, (infrared) remote control, camera, card reader, fax machine, dongle, biometric reader, microphone, touch screen, touchpad, trackball, stylus, scanner, storage device, transceiver, video device/source, etc. The output device 1812 may be a printer, fax machine, video display (e.g., cathode ray tube (CRT), liquid crystal display (LCD), light-emitting diode (LED), plasma, Plasma display panel (PDP), Organic light-emitting diode display (OLED) or the like), audio speaker, etc.

In some embodiments, the processor 1802 may be disposed in communication with a communication network 1809 via a network interface 1803. The network interface 1803 may communicate with the communication network 1809. The network interface 1803 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. The communication network 1809 may include, without limitation, a direct interconnection, local area network (LAN), wide area network (WAN), wireless network (e.g., using Wireless Application Protocol), the Internet, etc. Using the network interface 1803 and the communication network 1809, the computer system 1800 may communicate with security system 1810.

In some embodiments, the processor 1802 may be disposed in communication with a memory 1805 (e.g., RAM, ROM, etc. not shown in FIG. 18) via a storage interface 1804. The storage interface 1804 may connect to memory 1805 including, without limitation, memory drives, removable disc drives, etc., employing connection protocols such as serial advanced technology attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 1805 may store a collection of program or database components, including, without limitation, user interface application 1806, an operating system 1807, web server 1808 etc. In some embodiments, computer system 1800 may store user/application data 1806, such as the data, variables, records, etc. as described in this disclosure. Such databases may be implemented as fault-tolerant, relational, scalable, secure databases such as Oracle or Sybase.

The operating system 1807 may facilitate resource management and operation of the computer system 1800. Examples of operating systems include, without limitation, Apple Macintosh OS X, Unix, Unix-like system distributions (e.g., Berkeley Software Distribution (BSD), FreeB SD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. User interface 1817 may facilitate display, execution, interaction, manipulation, or operation of program components through textual or graphical facilities. For example, user interfaces may provide computer interaction interface elements on a display system operatively connected to the computer system 1800, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, Javascript, AJAX, HTML, Adobe Flash, etc.), or the like.

In some embodiments, the computer system 1800 may implement a web browser 1808stored program component. The web browser may be a hypertext viewing application, such as Microsoft Internet Explorer, Google Chrome, Mozilla Firefox, Apple Safari, etc. Secure web browsing may be provided using HTTPS (secure hypertext transport protocol), secure sockets layer (SSL), Transport Layer Security (TLS), etc. Web browsers may utilize facilities such as AJAX, DHTML, Adobe Flash, JavaScript, Java, application programming interfaces (APIs), etc. In some embodiments, the computer system 1800 may implement a mail server 1819 stored program component. The mail server may be an Internet mail server such as Microsoft Exchange, or the like. The mail server may utilize facilities such as ASP, ActiveX, ANSI C++/C#, Microsoft .NET, CGI scripts, Java, JavaScript, PERL, PHP, Python, WebObjects, etc. The mail server may utilize communication protocols such as Internet Message Access Protocol (IMAP), Messaging Application Programming Interface (MAPI), Microsoft Exchange, Post Office Protocol (POP), Simple Mail Transfer Protocol (SMTP), or the like. In some embodiments, the computer system 1800 may implement a mail client stored program component. The mail client may be a mail viewing application, such as Apple Mail, Microsoft Entourage, Microsoft Outlook, Mozilla Thunderbird, etc.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include Random Access Memory (RAM), Read-Only Memory (ROM), volatile memory, nonvolatile memory, hard drives, CD ROMs, DVDs, flash drives, disks, and any other known physical storage media.

Advantages of the embodiment of the present disclosure are illustrated herein.

In an embodiment, the positions for imaging are recognized by the user and the computing unit without immediate reference to what the imaging device sees. Therefore, it is not necessary to illuminate the retina for navigational purposes, or to train the user in recognition of retinal features. This reduces total incident light, and simplifies the provision of a wider pool of users qualified to obtain images for screening purposes.

In an embodiment, by stillness of the device, by the choice of still photography, and by the immediate quality control, the captured images are superior to those extracted from a video record.

In an embodiment, the operator selected still image enables to focus the image suitably before acquiring and storing in a definite ordered sequence. This makes it easier for image stitching while making the wide angle image.

In an embodiment, the present disclosure provides reduced storage requirement compared to the current video equipment for any type of compression.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor and a processor capable of processing and executing the queries. A non-transitory computer readable medium may comprise media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. Further, non-transitory computer-readable media comprise all computer-readable media except for a transitory. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as an optical fiber, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory computer readable medium at the receiving and transmitting stations or devices. An “article of manufacture” comprises non-transitory computer readable medium, hardware logic, and/or transmission signals in which code may be implemented. A device in which the code implementing the described embodiments of operations is encoded may comprise a computer readable medium or hardware logic. Of course, those skilled in the art will recognize that many modifications may be made to this configuration without departing from the scope of the invention, and that the article of manufacture may comprise suitable information bearing medium known in the art.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIG. 4 show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processing unit or by distributed processing units.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Referral Numerals:

| Reference | |
|---|---|
| Number | Description |
| 200 | Imaging Device |
| 203 | Tip |
| 205 | Light delivering means |
| 207 | Light collecting means |
| 209 | Image sensor |
| 212 | Computing Unit |
| 215 | Display Unit |
| 220 | Processor |
| 222 | Memory |
| 224 | Interface |
| 230 | Set of preferred directions |
| 270 | Curved reflective surface |
| 1800 | Computer System |
| 1801 | I/O Interface |
| 1802 | Processor |
| 1803 | Network Interface |
| 1804 | Storage Interface |
| 1805 | Memory |
| 1806 | User Interface Application |
| 1807 | Operating System |
| 1808 | Web Browser |
| 1810 | Security System |
| 1811 | Input Device |
| 1812 | Output Device |

1.-10. (canceled)

11. A method for imaging of wider angle fundus of an eye of a subject, the method comprising:
providing, by a computing unit, guidance to a user, moving an imaging device, for placement of the imaging device at a set of predetermined locations on the eye;
receiving, by the computing unit, a set of images of the retina of the eye, upon determining position of the imaging device to be on at least one of the set of predetermined locations, wherein the set of images are captured by the imaging device upon receiving instructions from the computing unit;
determining, by the computing unit, quality of the set of images, to obtain an acceptable image for each of the set of the predetermined locations, wherein the guidance is provided to focus the imaging device on each of the at least one of the set of predetermined location until an acceptable image is obtained; and
concatenating, by the computing unit, the acceptable images of each of the set of predetermined locations, to provide a wider-angle target image of the fundus of the eye.

12. The method as claimed in claim 11, wherein the set of predetermined locations on the eye are vertices of a polyhedron.

13. The method as claimed in claim 11, wherein guiding the placement of the imaging device comprises providing a notification to the user on a display unit of the imaging device.

14. The method as claimed in claim 13, wherein guiding the placement of the imaging device comprises providing a feedback upon determining the imaging device to be away from the set of predetermined locations.

15. The method as claimed in claim 11, wherein the set of images at predetermined locations are captured by the imaging device by:
providing, by a light emitting unit of the imaging device, light to retina through pupil of the eye;
receiving, by a light collecting unit the imaging device, light reflected from the fundus of the eye; and
capturing, by an image sensor of the imaging device, the set of images using the reflected light.

16. The method as claimed in claim 11, wherein concatenating of the acceptable images to create the wider angle target retina image of the eye is performed by:
determining a plurality of points in the acceptable images of each of the set of predetermined locations, to be matching with each other; and
concatenating the acceptable image of each of the set of predetermined locations using the matched plurality of points.

17. A computing unit for imaging of wider angle fundus of an eye of a subject, comprising:
a processor; and
a memory communicatively coupled to the processor, wherein the memory stores processor-executable instructions, which, on execution, causes the processor to:
provide guidance to a user moving an imaging device, for placement of the imaging device at a set of predetermined locations on the eye;
receive a set of images of the retina of the eye upon determining position of the imaging device to be on at least one of the set of predetermined locations, wherein the set of images are captured by the imaging device upon receiving instructions from the computing unit;
determine quality of the set of images, to obtain an acceptable image for each of the set of the predetermined locations, wherein the guidance is provided to focus the imaging device on each of the at least one of the set of predetermined location until an acceptable image is obtained; and
concatenate the acceptable image of each of the set of predetermined locations to provide a wider-angle target image of the fundus of the eye.

18. The computing unit as claimed in claim 11, wherein the set of predetermined locations on the eye are vertices of a polyhedron

19. The computing unit as claimed in claim 17, wherein guiding the placement of the imaging device comprises providing a notification to the user on a display unit of the imaging device.

20. The computing unit as claimed in claim 17, wherein guiding the placement of the imaging device comprises providing a feedback upon determining at least one of, the imaging device to be away from the set of predetermined locations.

21. The computing unit as claimed in claim 17, wherein the set of images are captured by the imaging device by:
providing, by a light emitting unit of the imaging device, light to retina through pupil of the eye;
receiving, by a light collecting unit the imaging device, light reflected from the retina of the eye; and
capturing, by an image sensor of the imaging device, the set of images using the reflected light.

22. The computing unit as claimed in claim 17, wherein concatenating of the acceptable image to provide the wider-angle target image of fundus of the eye is performed by:
determining a plurality of points in the acceptable image of each of the set of predetermined locations, to be matching with each other; and
concatenating the acceptable image of each of the set of predetermined locations using the matched plurality of points.
